(19) United States
(12) Patent Application Publication
(10) Pub. No.: US 2007/0010691 A1
(43) Pub. Date: Jan. 11, 2007

(54) Enantioselective synthesis of enantiomerically enriched compounds

(75) Inventors: Oress Piccolo, Sirtori (IT); Fausta Ulgheri, Li Punti (IT); Mauro Marchetti, Bonnanaro (Sassari) (IT)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: CONSIGLIO NAZIONALE DELLE RICERCHE, Roma (IT)

(21) Appl. No.: 10/562,813

(22) PCT Filed: Jul. 1, 2004
(86) PCT No.: PCT/EP04/07193

§ 371 Date: Jun. 14, 2006

(30) Foreign Application Priority Data
Jul. 2, 2003 (IT) MI2003 A 001354

(51) Int. Cl. C07C 237/10

(52) U.S. Cl. 564/123; 562/401

(57) ABSTRACT

Method of preparing an enantiomerically enriched compound of formula (II) comprising enantioselective hydrogenation of a compound of general formula (I): where W, X and Z have the meanings indicated in the description, to give a compound of general formula (II): where W, Y, T and C* have the meanings indicated in the description, in the presence of a catalyst or its suitable precursor based on Rh, Ru or Ir, having an oxidation state of 0, +1 or +2, and containing at least one enantiomerically enriched chiral ligand.

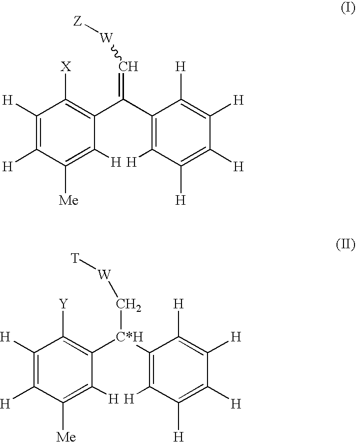

The present invention relates to a new method of synthesis of enantiomerically enriched compounds.

(R)-tolterodine of Formula (T) 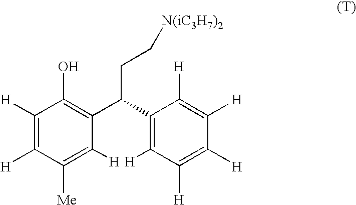
in the form of salt of tartaric acid, has recently been launched successfully on the world market as a drug against urinary incontinence.

Moreover, document U.S. Pat. No. 6,310,103 describes the corresponding enantiomer (S)-tolterodine of formula (T′) and its salts as drugs for use in the treatment of disorders of the urinary and gastrointestinal tracts.

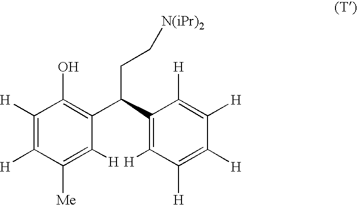

The methods of synthesis for the production of (R,S)-tolterodine, its enantiomers and the corresponding salts described in patent EP-A-0 325 571 include numerous steps (at least 6). Some of these steps involve the use of toxic or dangerous reagents and solvents and often give low yields. Moreover, production of the pure enantiomer, which is the pharmacologically active principle, employs separation by formation of diastereomeric salts which, by its nature, can only give a yield below 50%.

A person skilled in the art will be aware that to reduce the production costs it would be useful to recover the (S) enantiomer by repeated racemizations and separations, but to the best of our knowledge a method of this type has never been described. Alternatively, it would be useful to find a method of synthesis that can lead to a finished product that is already enantiomerically pure or at least substantially enriched in the desired enantiomer. Apparently, this has been the object of intensive research.

Patent application WO 0149649, equivalent to the already cited U.S. Pat. No. 6,310,248, describes a synthetic route that leads to an enantiomerically enriched 4-phenyl-6-methyl-chroman-2-one [(II); Y+T=O], with an enantiomeric excess (e.e.) of 89%. According to that document, the chromanone in the example had an absolute (S) configuration and could be converted to tolterodine enantiomerically enriched in the (R) enantiomer by known methods. In fact, according to the present inventors, the said chromanone should lead to tolterodine enantiomerically enriched in the (S) enantiomer. It can, however, be conjectured that changing the absolute configuration of the chiral reagent used (for example (S)-MeCBS instead of (R)-MeCBS) might lead to the (R) enantiomer. Nevertheless, this method also involves numerous steps and the use, as chiral reagent, of a boron derivative (MeCBS) that is expensive and not very suitable for production on an industrial scale.

Not even the synthesis described in J. Org. Chem. 63, 8067 (1998) is without drawbacks, because it uses reagents that are difficult to handle on a large scale and also involves the use of a chiral auxiliary that must then be recovered and recycled. Moreover, that article also describes the difficulties inherent in the synthesis of tolterodine or its suitable precursors by asymmetric hydrogenation (page 8067, left column, second paragraph), thus dissuading a person skilled in the art from this synthetic route.

Some of the present inventors have also investigated a method of hydroformylation reported in Org. Process Res. & Developm. 6, 379 (2002); however, this reaction, which proved industrially advantageous for producing the racemic product (R,S)-tolterodine, gave rather unsatisfactory results in enantioselective synthesis, which were also confirmed by the results recently reported in patent application WO 0204399. In fact tolterodine or its precursors are obtained with e.e. <10%, with negligible enrichment in the desired enantiomer.

Finally, a recent article [Tetrahedron Letters, 40, 3293 (1999)] described an asymmetric hydrogenation, in the presence of chiral diphosphinic catalysts of Rh or Ru, of alkaline or alkylammonium salts of suitable 3,3-diaryl acrylic acids to obtain 4-arylcoumarines. In that article it is emphasized that good enantiomeric excess (e.e.) is only obtained on particular, appropriately substituted substrates and in particular reaction conditions.

In its turn, the asymmetric hydrogenation of substituted 3,3-diaryl allylic alcohols described in Tetrahedron Asymmetry, 6, 835 (1995) has the drawback that it involves reaction times of several days and the use of quite high hydrogen pressures. Therefore it cannot be used industrially.

Surprisingly, the present inventors have now found an asymmetric synthetic route that does not have the aforesaid shortcomings and is based on a reaction of hydrogenation in the presence of a catalyst based on Rh, Ru or Ir, having an oxidation state of 0, +1 or +2, and containing at least one chiral ligand.

In one of its aspects the present invention therefore relates to a method of preparing an enantiomerically enriched compound of formula (II), characterized in that it comprises the enantioselective hydrogenation of a compound of general formula (I): 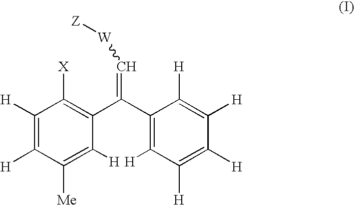
where

W is a CH₂ group or a C═O group;

X is a hydroxy, C₁-C₆ alkoxy, benzyloxy, C₁-C₆ acyloxy, O-tetrahydropyranyl, O-tetrahydrofuryl group, a group O⁻M⁺ in which M⁺ is a cation of an alkali metal or a cation N⁺R₁R₂R₃ where R₁, R₂ and R₃, which may be identical or different, are a C₁-C₈ alkyl, C₃-C₈ cycloalkyl or benzyl group;

Z, when W is CH₂, is a hydroxy group whereas, when W is C═O, it is a hydroxy, C₁-C₆ alkoxy, benzyloxy or N(iC₃H₇)₂ group, a group O⁻M⁺ in which M⁺ is a cation of an alkali metal or a cation N⁺R₁R₂R₃ where R₁, R₂ and R₃, which may be identical or different, are a C₁-C₈ alkyl, C₃-C₈ cycloalkyl or benzyl group;
to give a compound of general formula (II): 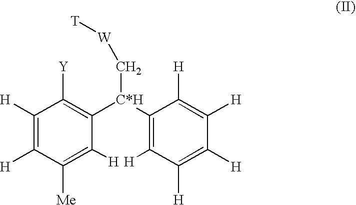
where

W has the meanings indicated above;

Y has the same meanings indicated above for X;

T has the same meanings indicated above for Z; or

when W is C═O

Y and T, together, are an oxygen atom; and

C* indicates the enantiomerically enriched chiral carbon atom;

in the presence of a catalyst or its suitable precursor based on Rh, Ru or Ir, having an oxidation state of 0, +1 or +2, and containing at least one enantiomerically enriched chiral ligand.

In a particularly preferred embodiment, the method of the present invention also includes the conversion of the compound of formula (II) thus obtained, in which Y, W and T are not already OH, CH₂ and N(iC₃H₇) respectively, to tolterodine enantiomerically enriched in the desired enantiomer.

In the present description the term “precursor” of a catalyst indicates a compound that is transformed to the desired catalyst in the presence of hydrogen.

The enantioselective hydrogenation according to the present invention can be carried out advantageously in homogeneous phase or in multiphase conditions, for example solid-liquid, immiscible liquid-liquid.

The catalyst and/or its precursor can be used as they are or immobilized on a suitable inorganic or organic support, for example silica, heteropolyacids/silica, heteropolyacids/alumina, zeolites, resins containing sulphonic, phosphonic and similar groups.

Typically, the molar ratio between the catalyst, or its precursor, and the compound of formula (I) is between 1/10 and 1/30 000. Preferably the said ratio is between 1/10 and 1/10 000. Even more preferably it is between 1/100 and 1/5000.

Typical examples of enantiomerically enriched chiral ligands according to the present invention are the mono- and diphosphinic, mono- and diphosphitic, mono- and diaminophosphinic ligands, such as the ligands containing a monophosphinic group and a C₁-C₆ alkoxy, benzyloxy, oxazoline, pyrrolidine or piperidine group, a group NR₁R₂, where R₁ and R₂, which may be identical or different, are a C₁-C₈ alkyl, C₃-C₈ cycloalkyl or benzyl group, a group NHCOR₃ or NHSO₂R₃ where R₃ is a C₁-C₈ alkyl, phenyl or tolyl group.

If necessary, the valence state of the metal of the catalyst according to the present invention is supplemented by at least one ancillary co-ligand.

Examples of suitable catalysts according to the present invention are:

Ru(TMBTP)(OCOCF₃)₂; Ru(TMBTP)(p.cymene)I₂; Ru(TMBTP)(p.cymene)Cl₂; Ru(BINAP)(OCOCF₃)₂; Rh(COD)(Chiraphos)ClO₄; Rh(NBD) (Chiraphos)ClO₄; where TMBTP denotes 2,2′,5,5′tetramethyl,3,3′bis(di-phenylphosphine),4.4′bithiophene, BINAP denotes 2,2′bis(diphenyl-phosphine)1,1′binaphthyl, Chiraphos denotes 2,3 bis(diphenyl-phosphine)butane, COD denotes cyclooctadiene, and NBD denotes norbornadiene.

Advantageously, the enantioselective hydrogenation according to the present invention is carried out at a pressure of 1-100 bar and preferably of 1-20 bar. Typically, during hydrogenation, the temperature is 20-100° C. and, preferably, 20-60° C. In a preferred embodiment, hydrogenation is carried out in the presence of a suitable solvent or a suitable solvent mixture. Typical examples of suitable solvents are C₁-C₄ alcohols, tetrahydrofuran, methylene chloride, C₁-C₄ alkyl aromatics or C₆-C₁₀ alkanes and their mixtures with water.

In the compounds of formula (I), W is preferably a C═O group; X is, preferably, OH or O⁻M⁺ in which M⁺ has the meanings already indicated above; Z is, preferably, OH, N(iC₃H₇)₂ or O⁻M⁺ in which M⁺ has the meanings already indicated above.

In the compounds of formula (II), W is preferably a CH₂ or C═O group; Y is, preferably, OH or O⁻M⁺ in which M⁺ has the meanings already indicated above; T is OH, N(iC₃H₇)₂ or O⁻M⁺ in which M⁺ has the meanings already indicated above. An especially preferred meaning is that in which Y and T, together, represent an oxygen atom of the lactone of formula (IIA)

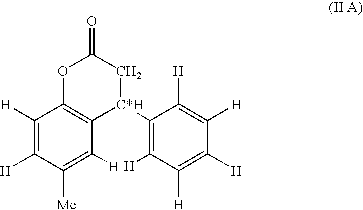

The compounds of formula (I) can be prepared by methods similar to those already known for preparing similar products. For example, when X═OH or O⁻M⁺, W is a C═O group and Z is a hydroxy, O⁻M⁺, alkoxy or N(iC₃H₇)₂ group; a convenient synthesis with high yield is that shown in Scheme 1. If necessary, this is then followed by treatment with a suitable base, for example an alkaline, ammoniacal hydroxide or a tetraalkylammonium hydroxide, to salify the acid group and the phenolic group.

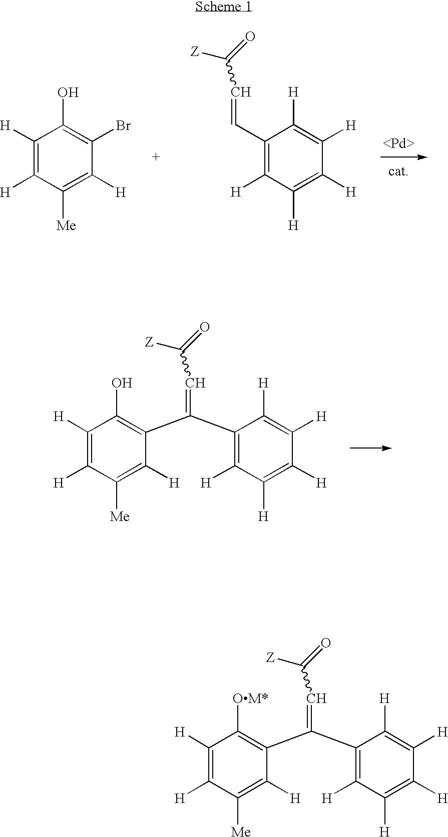

When the compound of formula (II) obtained by enantioselective hydrogenation is tolterodine (Y═OH, W═CH₂ and T=N(iC₃H₇)₂) enriched in the desired enantiomer, this is isolated by known techniques, for example by fractional crystallization of one of its salts, for example the tartrate, until the required pharmaceutical specifications are met.

However, when this is not tolterodine, the compound of formula (II) enriched in the desired enantiomer is easily converted to tolterodine by known techniques, for example those described in patents U.S. Pat. No. 5,922,914, WO 01/49 649 and EP-A-0 325 571 or by the techniques described in the following examples.

The following examples serve the purpose of illustrating the invention, though without limiting it in any way.

### EXAMPLE 1

### 6-methyl-4-phenyl-chromen-2-one

### (I; X+Z=O; W═CO)

2-Bromo-4-methylphenol (2.4 ml; 19.7 mmol), Et₄NCl (2.2 g; 13.3 mmol), Cy₂(Me)N (4.2 ml; 19.7 mmol) and Pd(OAc)₂ (59 mg; 0.26 mmol) were added under nitrogen at room temperature to a solution of methyl cinnamate (2.1 g; 13.1 mmol) in dimethylacetamide (40 ml). The reaction mixture was stirred at 95° C. for 48 h, then cooled and filtered on celite. The solution was diluted with Et₂O and washed 3 times with H₂O. The organic phase was dried over Na₂SO₄ and the solvent was evaporated under vacuum. GC-MS showed a conversion of 94%.

The raw reaction product was purified by flash chromatography. (SiO₂, n-hexane:Et₂O 7:3) and the fractions collected were crystallized from Et₂O/n-hexane to give pale yellow crystals (2.4 g; 77% yield). m.p.=132-134° C.

¹H NMR (400 MHz, CDCl₃) δ 2.34 (s, 3H, OCH₃), 6.36 (s, 1H, CH), 7.25-7.38 (m, 3H), 7.44-7.47 (m, 2H), 7.52-7.56 (m, 3H);

¹³C NMR (400 MHz, CDCl₃) δ 21.17; 115.43; 117.30; 118.917; 126.93; 128.662; 129.09; 129.82; 133.15; 134.11; 135.62; 152.55; 155.86; 161.24.

### EXAMPLE 2

### 6-methyl-4-phenyl-chroman-2-one (IIA)

A glass cylinder placed in a steel autoclave was loaded with 6-methyl-4-phenyl-chromen-2-one (1 g; 4.2 mmol), [Rh(COD)Cl]₂ (104.5 mg; 0.2 mmol), (S,S)-Chiraphos (180.8 mg; 0.4 mmol), CH₃OH (10 ml) and NaOH 4N (2.1 ml), then it was evacuated and the autoclave was pressurized to 12 bar with H₂. The reaction mixture was stirred at 50° C. for 24 h, then cooled to room temperature and the gas was removed. The solvent was removed in a rotary evaporator and the raw product, absorbed in H₂O, was washed with CH₂Cl₂ (2×30 ml), the aqueous phase was then acidified with 6N HCl to pH=1-2 and was then extracted with CH₂Cl₂ (30 ml×3). The organic phases were combined and dried over Na₂SO₄, filtered on celite and the solvent was evaporated at reduced pressure.

GC of the raw product (DetTBuSilβCDX column 25 m, carrier gas N₂, T initial=100° C., initial isotherm time=1, heating rate=2, T final=200° C., final isotherm time=10, flow 2, N₂ pressure=30 psi) showed that conversion was 96% and e.e. =80% enriched in the enantiomer at lower retention time to which the absolute (S) configuration was attributed [(S) enantiomer retention time=46.12 min, (R) enantiomer retention time=48.55 min, retention time of 6-methyl-4-phenyl-chromen-2-one=53.05 min]. ¹H NMR in CDCl₃ of the raw product showed that the product was a mixture of (IIA) and the corresponding uncyclized product (II; Y=T=OH and W═CO) in a ratio of 1:6, approximately, and that in time the open form cyclizes spontaneously and that this cyclization is complete when operating under reflux for 4 h in toluene in the presence of catalytic amounts of pTsOH acid.

The raw 6-methyl-4-phenyl-chroman-2-one (IIA) was purified by flash chromatography (SiO₂, hexane:Et₂O 7:3) to give 850 mg of a white solid (yield: 84%). Dissolving the product in hot CH₃OH and then cooling, 170 mg (yield: 20%) of white needles of product (S) (IIA) were obtained, with e.e. >99% [retention time=46.12 min], as determined by GC analysis; [α]_{D}²⁰=−2.8 (CHCl₃, c=1.44), m.p.=103-105° C.

¹H NMR (CDCl₃, 400 MHz), δ 2.26 (s, 3H); 2.99 (dd, J=6.4, 16.4 Hz, 1H); 3.06 (dd, J=6.4, 16.4 Hz, 1H); 4.30 (t, J=6.4 Hz, 1H); 6.78 (bs, 1H); 7.00-7.18 (m, 4H); 7.28-7.38 (m, 3H);

¹³C NMR (CDCl₃, 100.57 MHz), δ 21.24; 37.56; 41.14; 117.07; 125.52; 127.73; 127.81; 129.31; 129.50; 134.51; 140.68; 140.78; 167.98.

### EXAMPLES 3-7

### 6-methyl-4-phenyl-chroman-2-one (IIA)

Following the same procedure as in Example 2 but with different catalysts and different substrate/catalyst molar ratios, the results presented in Table 1 were obtained. The predominant enantiomer had the absolute (S) configuration.

TABLE 1

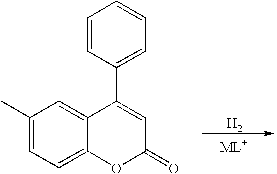

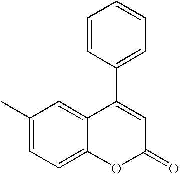

| | | | | | con- | |
|---|---|---|---|---|---|---|
| | | | | | ver- | |
| | | | | | sion | ee |
| ML | S/C | T | P | t | % | (%) |
| [Rh(COD)Cl₂(S,S)Chiraphos | 200/1 | 50° C. | 12 | 24 | 70% | 50% |
| | | | bar | h | | |
| | 2000/1 | 50° C. | 12 | 24 | 36% | 50% |
| | | | bar | h | | |
| [Rh(nbd)BF₄](S,S)Chiraphos | 1000/1 | 50° C. | 12 | 24 | 11% | 20% |
| | | | bar | h | | |
| [RU(II)-(S)-(−)- | 100/1 | 50° C. | 12 | 24 | 22% | 44% |
| BINAP(OAc)₂ | | | bar | h | | |
| [Ru(TFA)₂(+)-TMBTP] | 100/1 | 50° C. | 12 | 24 | 90% | 80% |
| | | | bar | h | | |

### EXAMPLE 8

### 6-methyl-4-phenyl-chroman-2-one (IIA)

Following the same procedure as in Example 2 but with the catalyst [Ru(TFA)₂(−)TMBTP] with a substrate/catalyst molar ratio of 100/1, a yield of 87% and e.e. of 81% were obtained after chromatographic purification. The predominant enantiomer had the (R) absolute configuration.

### EXAMPLE 9

### (S)-Tolterodine (formula T′)

Following procedure similar to that described in patent U.S. Pat. No. 5,922,914, a solution of 100 mg (0.42 mmol) of (IIA), having [α]_{D}²⁰=−2.8 (CHCl₃, c=1.44) and prepared according to the preceding Example 2, in anhydrous toluene (3 ml), was placed in a 100-ml two-necked flask that had been flamed beforehand. A solution of 1 M. DIBAL in toluene (440 μl, 0.44 mmol) was added dropwise to this solution, under N₂ and at −25° C.

The reaction was monitored by GC-MS and was stopped with 3 ml of ethyl acetate at −25° C. after 5 h, when GC-MS showed there was formation of 6-methyl-4-phenyl-chroman-2-ol at 89%, together with unreacted starting product (7%) and a product of further reduction [3-phenyl-3(2′hydroxy,5′methyl)phenyl-propan-1-ol] (4%). 3 ml of a 23% citric acid solution was added. The solution was stirred at room temperature over night. The organic phase was separated and washed with H₂O, dried over Na₂SO₄, filtered and the solvent was removed by evaporation at reduced pressure.

The raw product thus obtained was placed in a glass cylinder in an autoclave. CH₃OH (5 ml), Pd/C₅% (20 mg), (Prⁱ)₂NH (147 μl, 1.05 mmol) and H₂ were added at 5 atmospheres. The reaction was continued for 12 h at 48° C. The temperature was brought back to room temperature and the autoclave was depressurized by eliminating the gas. After filtration of the catalyst on celite, a GC-MS analysis was carried out, which showed 6-methyl-4-phenyl-chroman-2-ol (2%), (IIA) 5%, [3-phenyl-3(2′hydroxy,5′methyl)phenyl-propan-1-ol] (16%), and (S)-tolterodine (77%). The raw product was purified by flash chromatography on SiO₂ (hexane:EtOAc(7:3)/Et₃N 98:2) to give a colourless oil (100 mg; 73%); [α]_{D}²⁰=−23 (c=1.5; CH₃OH).

¹H NMR (CD₃OD, 400 MHz), δ 0.97 (d, J=2 Hz, 3H); 0.99 (d, J=2 Hz, 3H); 2.1-2.2 (m, 2H); 2.17 (s, 3H); 2.39-2.45 (m, 2H); 3.02 (m, 1H); 4.32 (t, J=7.6 Hz); 6.63 (d, J=7.8 Hz, 1H); 6.78 (dd, J=2.0, 8.2 Hz, 1H); 6.90 (d, J=2.3, 1H); 7.09-7.31 (m, 5H);

¹³C NMR (CD₃OD, 100.57 MHz), δ 20.32; 20.79; 37.48; 42.73; 45.95: 48.79; 116.26; 126.81; 128.27; 129.11; 129.24; 129.41; 132.47; 164.38; 153.74.

### EXAMPLE 10

### (S)-tolterodine D-tartrate

D-tartaric acid (34.5 mg; 0.23 mmol) was added to a solution of (S)-tolterodine (75 mg; 0.23 mmol), prepared according to the preceding Example 9, in EtOH (5 ml). The mixture thus obtained was heated to about 50° C., filtered while hot to remove a slight turbidity, and then concentrated to dryness at reduced pressure to give a white solid. m.p.=205-207° C.; [α]_{D}²⁵=−37 (c=1, CH₃OH).

### EXAMPLE 11

### (R)-tolterodine L-tartrate

Following a procedure similar to that described in the preceding Example 9 but starting from a sample of (R)-6-methyl-4-phenyl-chroman-2-one having e.e. 81%, obtained according to the preceding Example 8, (R)-tolterodine (T) was obtained at 70% yield.

The corresponding salt with L-tartaric acid, prepared and aspirated to dryness, had [α]_{D}²⁵=+29.1 (c=1, CH₃OH).

1. A method of preparing an enantiomerically enriched compound of formula (II), comprising enantioselectively hydrogenating a compound of general formula (I): 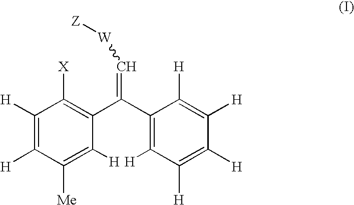
where
W is a CH₂ group or a C═O group;
X is a hydroxy, C₁-C₆ alkoxy, benzyloxy, C₁-C₆ acyloxy, O-tetrahydropyranyl, O-tetrahydrofuryl group, a group O⁻M⁺ in which M⁺ is a cation of an alkali metal or a cation N⁺R₁R₂R₃ where R₁, R₂ and R₃, which may be identical or different, are a C₁-C₈ alkyl, C₃-C₈ cycloalkyl or benzyl group;
Z, when W is CH₂, is a hydroxy group whereas, when W is C═O, it is a hydroxy, C₁-C₆ alkoxy, benzyloxy or N(iC₃H₇)₂ group, a group O⁻M⁺ in which M⁺ is a cation of an alkali metal or a cation N⁺R₁R₂R₃ where R₁, R₂ and R₃, which may be identical or different, are a C₁-C₈ alkyl, C₃-C₈ cycloalkyl or benzyl group;
to give a compound of general formula (II): 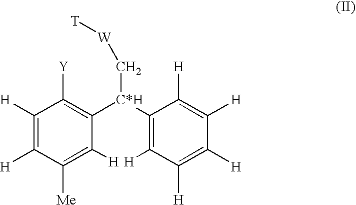
where
W has the meanings indicated above;
Y has the same meanings indicated above for X;
T has the same meanings indicated above for Z; or
when W is C═O
Y and T, together, are an oxygen atom; and
C* indicates the enantiomerically enriched chiral carbon atom;
in the presence of a catalyst or its suitable precursor based on Rh, Ru or Ir, having an oxidation state of 0, +1 or +2, and containing at least one enantiomerically enriched chiral ligand.

2. The method according to claim 1, wherein the compound of formula (II) in which Y, W and T are not OH, CH₂ and N(iC₃H₇)₂, respectively, is converted to tolterodine enantiomerically enriched in the desired enantiomer.

3. The method according to claim 1, wherein the method is carried out in homogeneous phase or in multiphase conditions.

4. The method according to claim 1, wherein the catalyst and its precursor are used as they are or immobilized on a suitable inorganic or organic support.

5. The method according to claim 4, wherein the support is at least one selected from the group consisting of silica, heteropolyacids/silica, heteropolyacids/alumina, zeolites, and resins containing sulphonic and phosphonic groups.

6. The method according to claim 1, wherein the molar ratio between the catalyst, or its precursor, and the compound of formula (I) is between 1/10 and 1/30000.

7. The method according to claim 6, wherein the molar ratio is between 1/10 and 1/10 000.

8. The method according to claim 6, wherein the molar ratio is between 1/100 and 1/5000.

9. The method according to claim 1, wherein the enantiomerically enriched chiral ligand is selected from mono- and diphosphinic, mono- and diphosphitic, mono- and diaminophosphinic ligands, ligands containing a monophosphinic group and a C₁-C₆ alkoxy, benzyloxy, oxazoline, pyrrolidine or piperidine group, a group NR₁R₂, where R₁ and R₂, which may be identical or different, are a C₁-C₈ alkyl, C₃-C₈ cycloalkyl or benzyl group, a group NHCOR₃ or NHSO₂R₃ where R₃ is a C₁-C₈ alkyl, phenyl or tolyl group.

10. The method according to claim 9, wherein optionally the valence state of the metal of the catalyst is supplemented with at least one ancillary co-ligand.

11. The method according to claim 10, wherein the catalyst is at least one selected from the group consisting of Ru(TMBTP)(OCOCF₃)₂; Ru(TMBTP)(p.cymene)I₂; Ru(TMBTP)(p.cymene)Cl₂; Ru(BINAP)(OCOCF₃)₂; Rh(COD) (Chiraphos)ClO₄; and Rh(NBD)(Chiraphos)ClO₄; where TMBTP denotes 2,2′,5,5′tetramethyl,3,3′bis(diphenylphosphine), 4.4′bithiophene, BINAP denotes 2,2′bis(diphenylphosphine)1,1′binaphthyl, Chiraphos denotes 2,3 bis(diphenylphosphine)butane, COD denotes cyclooctadiene, and NBD denotes norbornadiene.

12. The method according to claim 1, wherein the enantioselective hydrogenation is carried out at a pressure of 1-100 bar.

13. The method according to claim 12, wherein the pressure is 1-20 bar.

14. The method according to claim 1, wherein the enantioselective hydrogenation is carried out at a temperature of 20-100° C.

15. The method according to claim 14, wherein the temperature is 20-60° C.

16. The method according to claim 1, wherein enantioselective hydrogenation is carried out in the presence of a solvent or a solvent mixture.

17. The method according to claim 16, wherein the solvent is at least one selected from the group consisting of C₁-C₄ alcohols, tetrahydrofuran, methylene chloride, C₁-C₄ alkyl aromatics, C₆-C₁₀ alkanes and their mixtures with water.

18. The method according to claim 1, wherein in the compound of formula (I)
W is a C═O group;
X is OH or O⁻M⁺ in which M⁺ has the meanings already indicated above;
Z is OH, N(iC₃H₇)₂ or O⁻M⁺ in which M⁺ has the meanings already indicated above.

19. The method according to claim 1, wherein in the compound of formula (II)
W is a CH₂ or C═O group;
Y is OH or O⁻M⁺ in which M⁺ has the meanings already indicated above;
T is OH, N(iC₃H₇)₂ or O⁻M⁺ in which M⁺ has the meanings already indicated above.

20. The method according to claim 19, wherein Y and T, together, represent an oxygen atom of the lactone of formula (IIA) [chemical structure]
